(19) United States
(12) Patent Application Publication
(10) Pub. No.: US 2004/0038871 A1
(43) Pub. Date: Feb. 26, 2004

(54) Conjugates of aminodrugs

(76) Inventors: Daniela Fattori, Rome (IT); Paolo Nunzio Ingallinella, Rome (IT); Antonello Pessi, Rome (IT)

Correspondence Address: MERCK AND CO INC, P O BOX 2000, RAHWAY, NJ, 070650907

(21) Appl. No.: 10/296,282

(22) PCT Filed: May 18, 2001
(86) PCT No.: PCT/EP01/05797

(30) Foreign Application Priority Data
May 24, 2000 (GB) 0012718.3

(51) Int. Cl. A61K 38/14; C07K 9/00

(52) U.S. Cl. 514/8; 530/322

(57) ABSTRACT

The present invention provides a method of coupling an aminodrug (especially a cytotoxic drug, e.g. daunorubicin or doxorubicin) and a peptide to form an aminodrug-peptide conjugate, the method comprising attaching a linker to an amino group of the drug, the linker including an aldehyde or ketone carbonyl group (derived, for example, from levulinic acid or 5-oxopentanoic acid), and forming an oxime by reaction of the carbonyl group with an O-alkylhydroxylamine derivative of the peptide (obtained, for example, by reacting the peptide with aminooxyacetic acid); aminodrug-peptide conjugates obtainable from the described method are also provided, as also are pharmaceutical compositions comprising the conjugates, and methods of using the conjugates in therapeutic medication.

The present invention relates to conjugates of aminodrugs, such as cytotoxic drugs, in particular anthracycline antibiotics with peptides, to a method for their production and to the use of such conjugates in therapy.

The anthracycline antibiotics, which include daunorubicin and doxorubicin shown at (I) below, are widely used as antineoplastic agents in tumour treatment. However, toxic dose-related side effects, such as nephrotoxicity and cardiotoxicity, limit their clinical application. Different approaches have been adopted in order to increase their therapeutic index. One way of reducing the therapeutic dose is tumour targeting obtained by attaching the cytotoxic compound to carrier peptides which show affinity to the tumour tissue (W. Arap et al., Science, 1998, 279, 377-380). By reversing the above reasoning, another attractive application for peptide-anthracyclinone conjugates is the study of cell/tissue affinity of ligands selected from combinatorial peptide libraries, through monitoring the selectivity of cell killing by conjugates as a benchmark for success.

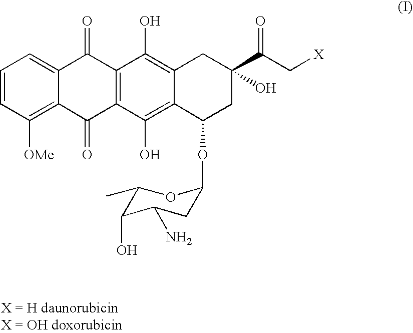

Several ways of conjugating these drugs to peptides have been published to date: formation of an amide bond on the sugar amino group (A. Trouet et al., Proc. Natl. Acad. Sci. U.S.A., 1982, 79, 626-629), formation of an ester bond on the primary hydroxyl of doxorubicin (A. Nagy et al., Proc. Natl. Acad. Sci. U.S.A., 1998, 95, 1794-1799), alkylation of the sugar amino group through reductive amination (D. Farquhar et al., J. Med. Chem., 1998, 41, 965-972), and introduction of a maleimide moiety for further ligation with a cysteine-containing peptide (Poster presentation at 3rd Lausanne Conference on Bioorganic Chemistry (1999), M. Langer et al.). Such methods, however, are not compatible with the entire variety of amino acid functionalities on the reacting peptide, so that some residues must be avoided, or selectively protected, with the ensuing solubility problems. Furthermore, particular problems are involved when attaching peptides to anthracyclines because the latter are very sensitive to acids, to bases, to oxidizing and to reducing agents. They also include rather reactive phenolic and alcoholic functions. So, for instance, the instability of the glycosidic bond to the acidic conditions normally used for N^{α} deprotection (Boc synthesis) or resin cleavage (Fmoc synthesis) precludes a solid phase approach to the problem.

It is known to link totally unprotected peptidic fragments by oxime ligation. Such a method is, for instance, described in: G. Tuchscherer, Tetrahedron Lett., 1993, 34, 8419-8422; K. J. Rose, J. Am. Chem. Soc., 1994, 116, 30-33; and L. E. Canne et al., J. Am. Chem. Soc., 1995, 117, 2998-3007. As described in these references a precursor O-alkylhydroxylamine is obtained by coupling (Boc-protected) aminooxyacetic acid, to a free amino group of one peptide.

In particular, the present inventors sought a general method to produce conjugates between aminodrugs, and in particular the anthracyclines, and a peptide of any sequence. The inventors appreciated that a precursor O-alkylhydroxylamine could be easily obtained by coupling an aminooxycarboxylic acid, such as aminooxyacetic acid, to a free amino group of the peptide. Therefore, their efforts were directed to introducing the partner carbonyl function into the aminodrug moiety. Although the anthracyclines already contain a ketone, modification of this carbonyl to form a methyl oxime has been shown to reduce cytotoxicity dramatically (K. Yamamoto et al., J. Med. Chem., 1972, 15, 872-875).

According to one aspect of the present invention there is provided a method of coupling an aminodrug, especially a cytotoxic drug, and a peptide to form an aminodrug-peptide conjugate, the method comprising attaching a linker to an amino group of the drug, the linker including an aldehyde or ketone carbonyl group, and forming an oxime by reaction of the carbonyl group with an O-alkylhydroxylamine derivative of the peptide.

Each component of the conjugate is considered in some more detail below.

Aminodrug

The aminodrug may be any which contains at least one free amino group. Preferably, the free amino group is not essential for activity. In general, preferred aminodrugs do not contain keto or aldehydo moieties or, if they do, these are unable, for instance because of the chosen reaction conditions, to compete effectively with the carbonyl group introduced through the linker. Preferred drugs for use in the method of the first aspect are cytotoxic drugs. Although they do contain an exocyclic keto group, particularly preferred cytotoxic drugs for use in the method of the first aspect are the anthracyclines of formula (II) set out below:

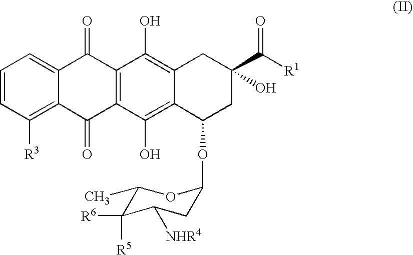

wherein:

R¹ is —CH₃, —CH₂OH, —CH₂OCO(CH₂)₃CH₃ or —CH₂OCOCH(OC₂H₅)₂;

R³ is —OCH₃, —OH or —H;

R⁴ is —H, benzyl, cyanomethyl or —CH(CN)CH₂(OMe);

R⁵ is —OH, —OTHP or —H; and

R⁶ is —OH or —H; provided that R⁶ is not —OH when R⁵ is —OH or —OTHP.

In this case, the linker is attached at the amino group of the sugar moiety.

Preferred examples of anthracycline antibiotics are set out in Table 1 below. Of these, daunorubicin and doxorubicin are most preferred.

TABLE 1

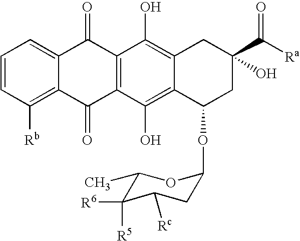

| Compound | R^{a} | R^{b} | R^{c} | R⁵ | R⁶ |
|---|---|---|---|---|---|
| daunorubicin^{a} | CH₃ | OCH₃ | NH₂ | OH | H |
| doxorubicin^{b} | CH₂OH | OCH₃ | NH₂ | OH | H |
| detorubicin | CH₂OCOCH(OC₂H₅)₂ | OCH₃ | NH₂ | OH | H |
| carminomycin | CH₃ | OH | NH₂ | OH | H |
| idarubicin | CH₃ | H | NH₂ | OH | H |
| epirubicin | CH₂OH | OCH₃ | NH₂ | OH | OH |
| esorubicin | CH₂OH | OCH₃ | NH₂ | H | H |
| THP | CH₂OH | OCH₃ | NH₂ | OTHP | H |

^{a}“daunomycin” is an alternative name for daunorubicin
^{b}“adriamycin” is an alternative name for doxorubicin

Although these compounds contain an exocyclic keto moiety which might be expected to compete with a carbonyl group provided on the linker, the inventors have found that, by appropriate choice of linker, and of reaction conditions for oxime formation, the selectivity of reaction at the carbonyl group of the linker can be increased.

Other possible cytotoxic drugs which may be coupled to peptides by the method of the present invention include:

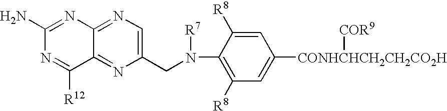

methotrexates of formula:

in which

R¹² is amino or hydroxy;

R⁷ is hydrogen or methyl;

R⁸ is hydrogen, fluoro, chloro, bromo or iodo; and

R⁹ is hydroxy or a moiety which completes a salt of the carboxylic acid;

mitomycins of formula:

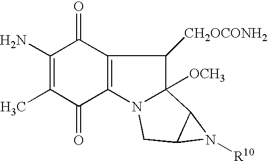

in which

R¹⁰ is hydrogen or methyl;

bleomycins of formula:

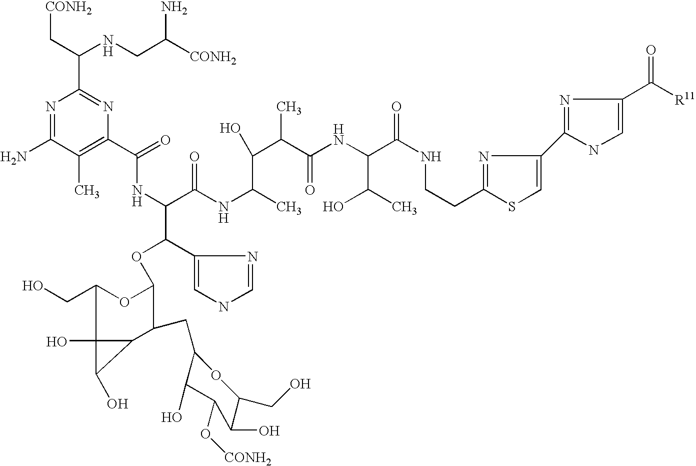

in which

R¹¹ is hydroxy, amino, C₁-C₃ alkylamino, di(C₁-C₃ alkyl)amino, C₄-C₆ polymethylene amino,

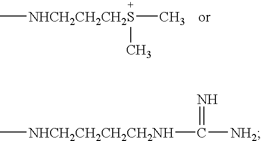

melphalan of formula:

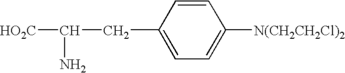

and analogues of thapsigargin such as are described in Bioorg. Med. Chem., 1999, 7, 1273-1280.

Linker

In general, the activity of the aminodrug, for instance the cytotoxic activity of the cytotoxic drug, in the intact conjugate of the drug and peptide should be greatly reduced or absent. However, the activity of the drug should increase significantly or be restored to the activity of the unmodified drug upon enzymatic cleavage of the conjugate at the target site of the drug. Consequently, the linker is preferably chosen such that it may be removed by enzymatic cleavage in vivo to release the drug or so that if it, or a part of it, remains attached to the drug after cleavage of the conjugate in vivo, then it does not significantly impair the activity of the drug.

The linker and the amino group of the drug may be joined in a variety of ways, for instance by forming a sulfonamido, urethane or urea linkage. However, the preferred method of attaching the linker and amino group is by formation of an amide bond.

Examples of suitable linkers, as attached to the amino group, are those of formula (III) below:

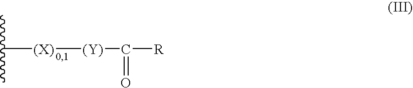

wherein X is selected from —CO—, —SO₂—, —SO₂NH—, —CO.O—, —CO.NH—, and —CR′R″— where each of R′ and R″ is independently selected from hydrogen and lower alkyl groups containing 1 to 10, preferably 1 to 6, particularly 1 to 3 carbon atoms.

In this formula, the group Y may be absent, but more preferably is an optionally substituted and/or interrupted alkylene group containing 1 to 6 carbon atoms, an optionally substituted and/or interrupted cycloalkylene group containing 3 to 7 carbon atoms, or an aromatic or heteroaromatic ring containing 2 to 10 carbon atoms. Preferably, Y is an unsubstituted and uninterrupted alkylene group. If substituted, the substituent is preferably one which enhances the electrophilicity of the carbonyl carbon atom. For instance, electron withdrawing groups at the carbon atom alpha to the carbonyl group, such as fluorine, may be tolerated. Generally substituents should be those which do not significantly reduce the reactivity of the carbonyl group and which are substantially unreactive towards the aminodrug and towards the peptide to be joined to it. Similar considerations apply to optional interrupting groups whose nature and position relative to the carbonyl group should be such that they do not reduce the reactivity of the carbonyl group, or result in undesirable side reactions. Additionally, such groups should not decrease the stability of the intact conjugate at sites in the body remote from the target site such as the general circulation. Typical interrupting groups include O, S, NH and N-(C₁₋₆)alkyl.

R in formula (III) is H or an optionally substituted and/or interrupted lower alkyl group containing 1 to 10, preferably 1 to 6, particularly 1 to 3 carbon atoms. Similar considerations apply to the choice of substituents and interrupting groups as were discussed above in respect of Y. In general, the group is preferably unsubstituted, although electron withdrawing groups, such as fluorine, may be tolerated at the position alpha to the carbonyl group.

A suitable linker may be selected depending on the drug to be included in the conjugate and may be joined to the amine group of the drug by methods well known to the person of skill in the art, e.g. by the formation of amide, sulfonamide, sulfamide, urethane or urea linkages.

Where the drug is an anthracycline, Y is preferably —CH₂CH₂— or —CH₂CH₂CH₂—, of which the latter is preferred. R is preferably a methyl group. X is preferably present and a carbonyl group.

Thus, the preferred linkers are those of formulae (IVa) and (IVb) below. Anthracycline derivatives including these linkers may be formed by reaction of the anthracycline of formula (II) with levulinic acid linker IVa) or 5-oxohexanoic acid linker IVb) to form anthracycline derivatives (Va) and (Vb) respectively:

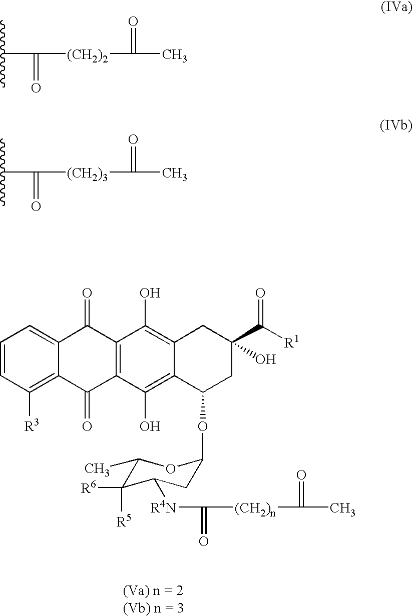

where R¹, R³, R⁴, R⁵ and R⁶ are as defined above.

Peptide

The peptide to be included in the conjugate is not particularly limited. The term should be understood broadly, to encompass short oligopeptides as well as polypeptides and proteins. For therapeutic applications, the peptide is preferably one displaying affinity for a target tissue at which a therapeutic effect is sought. For instance, it may be an antibody or antibody fragment capable of binding an antigen expressed on the surface of the tissue. Another possibility is that it is a protein which is recognised and bound by a receptor on the tissue surface. Yet another possibility is that it is a peptide which is preferentially degraded by enzymes present in the target tissue with resultant release of the drug. In the case of a cytotoxic drug, where the target is tumour tissue, the peptide may be one which is bound by tumour tissue (e.g. because it is recognised by a receptor which is overexpressed by tumour tissue), or because it is preferentially degraded by enzymes present in tumour tissue with resultant release of the cytotoxic drug. There are many possibilities, but examples of peptides previously suggested for targeting cytotoxic drugs, and which may be employed in the method of the present invention, include those subject to enzymatic degradation, for instance proteolytic cleavage by prostate specific antigen, such as those peptides described in WO 99/28345, WO 98/18493 and WO 97/12624 (all in the name of Merck & Co., Inc.), peptides able to target tumour vasculature, e.g. integrin binding peptides or peptides including a cell adhesion motif (see e.g. W Arap et al., Science, 1998, 279, 377-380), and somatostatin (see e.g. A. Nagy et al., Proc. Natl. Acad. Sci. U.S.A., 1998, 95, 1794-1799). Drug-peptide conjugates subject to degradation by enzymes such as proteases and peptidases are described in U.S. Pat. No. 4,703,107 (Monsigny et al.) and WO 96/05863 (La Region Wallonne et al.). The peptides described in those references may also be of utility in the present invention.

Additionally, as mentioned earlier, the peptide may be one from a library of peptides whose cell or tissue affinity is under investigation.

A further possibility is that the peptide is a carrier for a hapten drug, the drug and peptide being coupled as described above. The resulting conjugate may be used to generate antibodies to the drug which may be used, for instance, in immunoassay or affinity chromatography.

Peptides for use in the method of the first aspect may incorporate conventional protecting groups for amino acid residues such as Fmoc (9-fluorenylmethoxycarbonyl), tert-butyl, Pmc (2,2,5,7,8-pentamethylchroman-6-sulphonyl), Boc (tert-butoxycarbonyl), Alloc (allyloxycarbonyl) and Trt (trityl). However, the peptides are preferably unprotected.

Peptides for use in this aspect of the invention are used in the form of their O-alkylhydroxylamine derivatives. These may be represented by the following formula (VI):

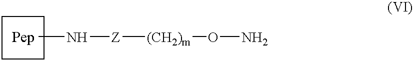

where

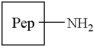

is an underivatised peptide with a free amino group; the group Z is selected from —CO— (forming an amide), —SO₂— (forming a sulfonamide), —CO.O— (forming a carbamate), —CO.NH— (forming a urea), and —SO₂.NH— (forming a sulfamide); and m is an integer from 1-6, and is preferably 1. The O-alkylhydroxylamine derivatives are formed by reaction of a free amino group of the peptide with a, preferably, protected aminooxyalkanoic acid such as protected aminooxyacetic acid. Suitable protecting groups for the aminooxy —NH₂ group will be apparent to those of skill in the art. Boc and Fmoc are typical examples.

The free amino group of the peptide which is reacted with the aminooxyalkanoic acid to form the O-alkylhydroxylamine may be the N-terminal amino group of the peptide or, alternatively, may be an amino group in the side chain of an amino acid residue of the peptide, e.g. lysine. Where more than one amino group of the peptide is available for reaction with aminooxyalkanoic acid it is preferable to protect those amino groups at which reaction with aminooxyalkanoic acid is undesired. After formation of the O-alkylhydroxylamine derivative it is preferred to remove all protecting groups prior to reaction with the drug-linker adduct.

The drug-linker adduct and the O-alkylhydroxylamine derivative of the peptide may be combined by standard conditions for oxime ligation, for instance as described in: G. Tuchscherer, Tetrahedron Lett., 1993, 34, 8419-8422; K. Rose, J. Am. Chem. Soc., 1994, 116, 30-33; and L. E. Canne et al., J. Am. Chem. Soc., 1995, 117, 2998-3007. Thus, the oxime may be formed in aqueous solution at a pH of around 4. However, the present inventors have found that, where the drug is an anthracycline so that reactive carbonyl groups are present in the drug and the linker, the selectivity of reaction with the linker carbonyl group can be improved by working at a somewhat higher pH. The preferred pH range for these compounds is from 5 to 7 and the pH is preferably around 6. As necessary, the desired oxime derivative may be separated by chromatographic techniques known to those in the art, such as HPLC.

By way of illustration only, a preferred scheme for coupling daunorubicin or doxorubicin and a peptide is illustrated below. By suitable manipulation of the reaction conditions, as discussed above, the proportion of the desired products, designated (B), can be maximised relative to the undesired products, designated (A).

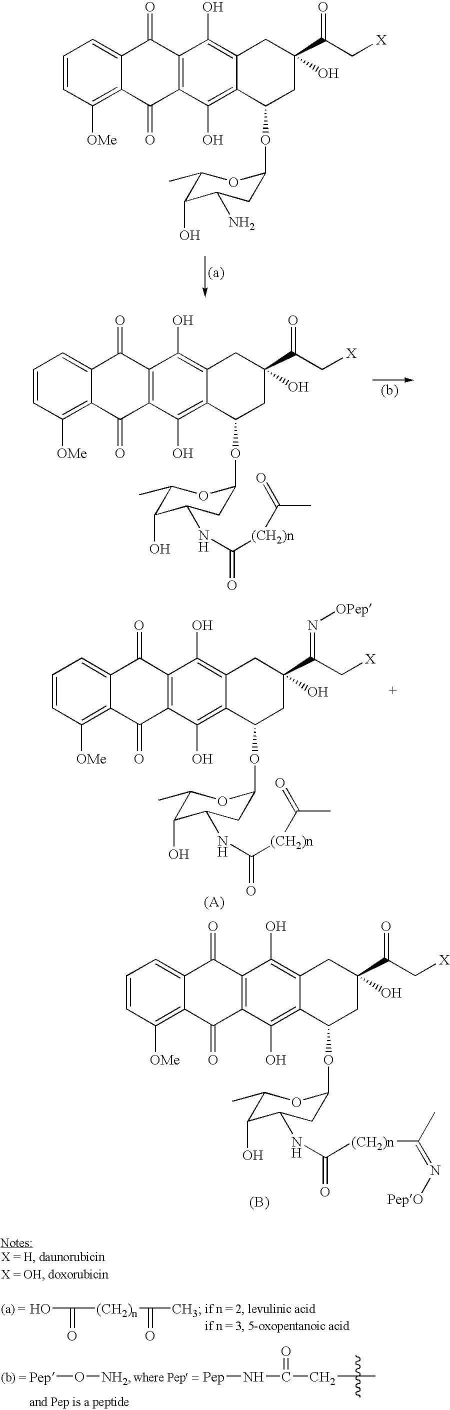

According to a second aspect of the invention there are provided conjugates of cytotoxic drugs and peptides as obtainable by the method of the first aspect.

These conjugates may be administered to a human or animal patient in the form of a pharmaceutical composition which comprises a conjugate and a pharmaceutically acceptable carrier, excipient or diluent therefor. As used herein, “pharmaceutically acceptable” refers to those agents which are useful in the treatment or diagnosis of a warm-blooded animal including, for example, a human, equine, porcine, bovine, murine, canine, feline or other mammal, as well as an avian or other warm-blooded animal. The preferred mode of administration is parenterally, particularly by the intravenous, intramuscular, subcutaneous, intraperitoneal, or intralymphatic route. Such formulations can be prepared using carriers, diluents or excipients familiar to one skilled in the art. In this regard, see, e.g., Remington's Pharmaceutical Sciences, 16th ed., 1980, Mack Publishing Company, edited by Osol et al. Such compositions may include proteins, such as serum proteins, for example human serum albumin, buffers or buffering substances such as phosphates, other salts, or electrolytes, and the like. Suitable diluents may include, for example, sterile water, isotonic saline, dilute aqueous dextrose, a polyhydric alcohol or mixtures of such alcohols, for example glycerin, propylene glycol polyethylene glycol, and the like. The compositions may contain preservatives such as phenethyl alcohol, methyl and propyl parabens, thimerosal, and the like. If desired, the composition can include about 0.05 to about 0.20 percent by weight of an antioxidant such as sodium metabisulfite or sodium bisulfite.

For intravenous administration, the composition preferably will be prepared so that the amount administered to the patient will be from about 0.01 to about 1 g of the conjugate. Preferably, the amount administered will be in the range of about 0.2 g to about 1 g of the conjugate. The conjugates of the invention are effective over a wide dosage range depending on factors such as the disease state to be treated or the biological effect to be modified, the manner in which the conjugate is administered, the age, weight and condition of the patient, as well as other factors to be determined by the treating physician. Thus, the amount administered to any given patient, must be determined on an individual basis.

Embodiments of the present invention are described below, by way of example only, and with reference to the accompanying drawings of which:

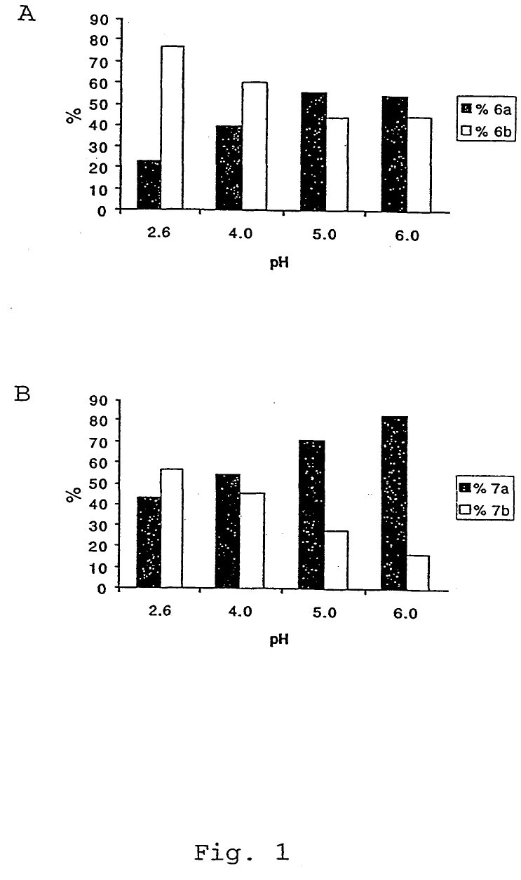

FIGS. 1A and 1B show the relative amounts of desired (▪) and undesired (□) product when a test peptide is coupled to daunorubicin using a levulinic acid linker (FIG. 1A) or a 5-oxopentanoic acid linker (FIG. 1B);

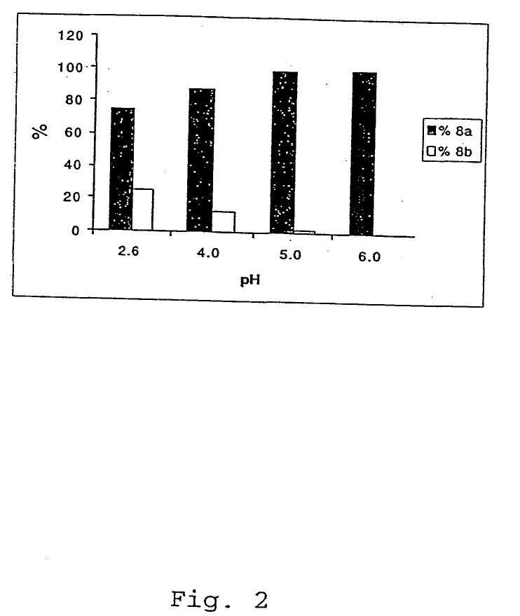

FIG. 2 shows the relative amounts of desired (▪) and undesired (□) product when a test peptide is coupled to doxorubicin using a 5-oxopentanoic acid linker.

### EXPERIMENTAL

Abbreviations

Alloc: allyloxycarbonyl

Boc: tert-butoxycarbonyl

MeCN: acetonitrile

DCM: dichloromethane

DIEA: diisopropylethylamine

DMF: N,N′-dimethylformamide

DMSO: dimethylsulfoxide

DIPC: diisopropylcarbodiimide

Fmoc: 9-fluorenylmethoxycarbonyl

HOBt: N-hydroxybenzotriazole

HOAt: 1-hydroxy-7-azabenzotriazole

MeOH: methanol

MTBE: methyl tert-butyl ether

Pmc: 2,2,5,7,8-pentamethylchroman-6-sulfonyl

PyBOP: benzotriazol-1-yloxytripyrrolidinophosphonium hexafluorophosphate

TFA: trifluoroacetic acid

Trt: trityl

General Methods

All the materials were obtained from commercial suppliers and used without further purification.

Thin layer chromatography (TLC) was performed on silica gel 60 F₂₅₄ precoated plates (Merck, Darmstadt). Analytical HPLC was performed on a Beckman System Gold chromatograph equipped with a diode-array detector and a Beckmann C-18 column (250×4.6 mm, 5 μm), operating flow rate 1 ml min⁻¹. Preparative HPLC was performed on a Waters 600E chromatograph equipped with a Jasco TV-975 detector (monitoring wavelength, 254 nm and 214 nm), Waters Delta-Pak™ C-18 column (100×250 mm, 15 μm). The operating flow rate was 30 ml min⁻¹. The solvent system was: eluent A, water (0.1% TFA); eluent B, MeCN (0.1% TFA). NMR spectra were recorded on a Brucker instrument operating at 400 MHz (¹H). Chemical shifts are reported in ppm relative to the solvent residual signal.

### Synthesis of N-Levulinate of Daunorubicin (2)

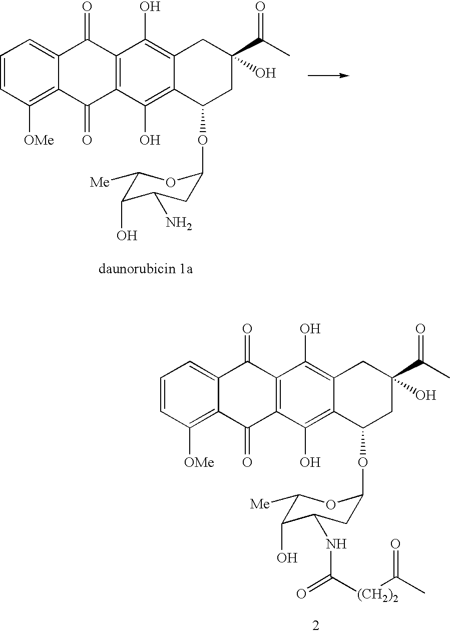

A solution of daunorubicin hydrochloride (56 mg, 0.10 mmol), levulinic acid (titer 97%, 12 mg, 0.10 mmol), PyBOP (52 mg, 0.10 mmol), HOBt (16 mg, 0.10 mmol) and DIEA (35 μl, 0.20 mmol) in DMF (0.5 ml) was stirred at room temperature. At the end of the reaction (monitored by TLC, silica, DCM/MeOH 9:1) the solution was diluted with DCM (5 ml) and washed with 1N HCl_{aq} (3 times), ss NaHCO₃ and brine, dried over Na₂SO₄ and concentrated to obtain a red oil. Chromatographic purification (silica, DCM-DCM/MeOH 9:1) afforded 35 mg (56%) of 2.

¹H-NMR (DMSO-⁶d): 7.94 (m, 2H), 7.64 (m, 1H), 7.52 (d, 1H), 5.52 (s, 1H), 5.22 (dd, 1H), 4.96 (dd, 1H), 4.72 (d, 1H), 4.17 (m, 1H), 3.99 (s, 3H), 3.99 (m, 1H), 3.37 (m, 1H), 3.00 (m, 3H), 2.57 (m, 2H), 2.25 (s, 3H), 2.17 (m, 2H), 2.05 (s, 3H), 1.84 (m, 1H), 1.74 (m, 2H), 1.42 (m, 1H), 1.13 (dd, 3H). ES-MS analysis: [M+H⁺] m/z=626, expected for C₃₂H₃₅NO₁₂ 625.

### Synthesis of N-5-oxopentanoate of Daunorubicin (3)

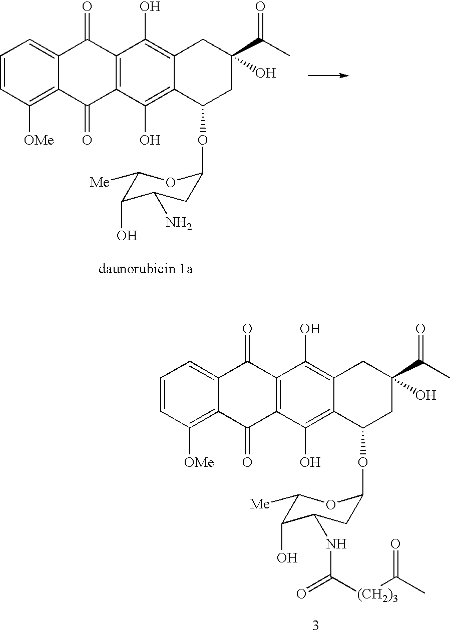

A solution of daunorubicin hydrochloride (56 mg, 0.10 mmol), 5-oxopentanoic acid (titer 97%, 15 mg, 0.11 mmol), PyBOP (57 mg, 0.11 mmol), HOBt (23 mg, 0.15 mmol) and DIEA (35 μl, 0.20 mmol) in DMF (0.5 ml) was stirred at room temperature. At the end of the reaction (monitored by TLC, silica, DCM/MeOH 9:1) the solution was diluted with DCM (5 ml) and washed with 1N HCl_{aq} (3 times), ss NaHCO₃ and brine, dried over Na₂SO₄ and concentrated to obtain a red oil. Chromatographic purification (silica, DCM-DCM/MeOH 9:1) afforded 36 mg (57%) of 3.

¹H-NMR (DMSO-⁶d): 7.89 (m, 2H), 7.66 (m, 1H), 7.47 (d, 1H), 5.53 (s, 1H), 5.21 (dd, 1H), 4.95 (dd, 1H), 4.71 (d, 1H), 4.16 (m, 1H), 4.00 (s, 3H), 3.39 (m, 1H), 3.37 (m, 1H), 3.00 (m, 2H), 2.37 (m, 2H), 2.26 (s, 3H), 2.03 (m, 2H), 2.03 (s, 3H), 1.84 (m, 1H), 1.74 (m, 2H), 1.63 (m, 2H), 1.42 (m, 2H), 1.15 (dd, 3H). ES-MS analysis: [M+H⁺] m/z=640, calculated for C₃₃H₃₇NO₁₂ 639.

### Synthesis of N-5-oxopentanoate of Doxorubicin (4)

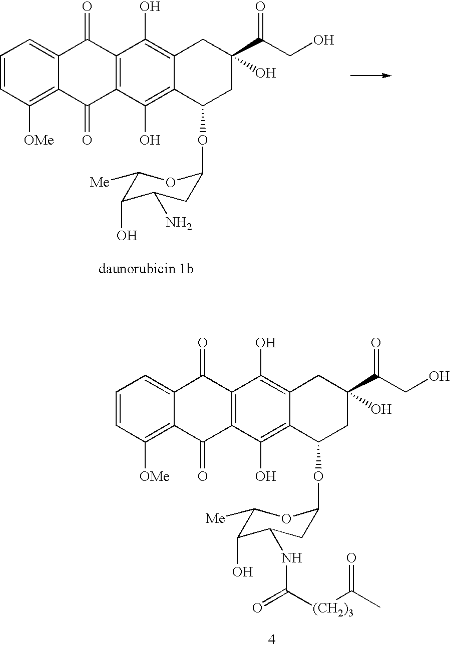

A solution of doxorubicin hydrochloride (174 mg, 0.30 mmol), PyBOP (171 mg, 0.30 mmol), HOBt (69 mg, 0.45 mmol), 5-oxopentanoic acid (45 mg, 0.33 mol), DIEA (105 μl, 0.60 mmol) in DMF (3 ml) was stirred at room temperature. At the end of the reaction (monitored by TLC, silica, DCM/MeOH 8:2) the solution was diluted with DCM (15 ml) and washed with water, 1N HCl_{aq} (3 times), ss NaHCO₃, water and brine, dried over Na₂SO₄ and concentrated to obtain 143 mg (72%) of 4.

¹H-NMR (DMSO-⁶d): 7.95 (m, 2H), 7.67 (m, 1H), 7.49 (d, 1H), 5.47 (s, 1H), 5.24 (dd, 1H), 4.97 (m, 1H), 4.85 (dd, 1H), 4.72 (d, 1H), 4.57 (m, 2H), 4.17 (m, 1H), 4.00 (s, 3H), 4.00 (m, 1H), 3.40 (m, 1H), 3.00 (m, 2H), 2.37 (m, 2H), 2.35-2.17 (m, 4H), 2.05 (s, 3H), 1.82 (m, 1H), 1.75 (m, 2H), 1.62 (m, 2H), 1.45 (m, 1H), 1.12 (dd, 3H). ES-MS analysis: [M+H⁺] m/z=656, calculated for C₃₃H₃₇NO₁₃ 655.

### Synthesis of Model Peptide Conjugates: Synthesis of N-aminooxyacetate of H-Ala-Tyr-Gly-NH₂ (5)

The peptide was synthesized by Fmoc-t-Bu chemistry on a Millipore 9050 Plus synthesizer on 0.5 g of Fmoc-PAL-PEG-PS resin 0.19 meq/g (PE PerSeptive). Side-chain protection for tyrosine was Fmoc-Tyr(tert-butyl)-OH. The protected amino acid (1 eq) was preactivated with PyBOP (1 eq), HOBt (1 eq), and DIEA (2 eq) using a 5-fold excess of acylant over the resin amino groups. Coupling times were 60 min. The N-terminus of the Ala was reacted with Boc-aminooxyacetic acid (1 eq), DIPC (1 eq) and HOBt (1 eq) for 2 h (5-fold excess of acylant). At the end of the assembly the resin was washed with DMF, MeOH, diethyl ether and dried in vacuo. The peptide resin was treated with 20 ml of TFA 88%, phenol 5%, triisopropylsilane 2%, water 5% (Reagent B) for 2 h. The resin was filtered and rinsed with TFA. The TFA solution was added dropwise to screw cap centrifuge tubes containing cold MTBE with a TFA/MTBE ratio of 1/10; after centrifugation at 3200×g (30 min), the ether solution was removed and the peptide precipitate resuspended in 50 ml of MTBE: the process was repeated twice. The dried precipitate was dissolved in MeCN/water and lyophilized.

The crude residue was purified by preparative HPLC, using isocratic elution (5% eluent B) followed by a linear gradient 5%-15% eluent B over 20 min.

¹H-NMR (DMSO-⁶d) (for the trifluoroacetate salt of 5): 10.50 (s, 1H), 8.20 (m, 2H), 8.05 (d, 1H), 8.00 (d, 1H), 7.00 (d, 2H), 6.60 (d, 2H), 4.40 (m, 1H), 4.30 (m, 1H), 4.35 (d, 2H), 3.70 (S_{br}, 2H), 2.90 (dd, 1H), 2.65 (dd, 1H), 1.15 (d, 3H). ES-MS analysis: [M+H⁺] m/z=383, calculated for C₁₆H₂₃N₅O₆ 382.

### General Procedure for the Analysis of the pH Dependence of Ligation Regioselectivity

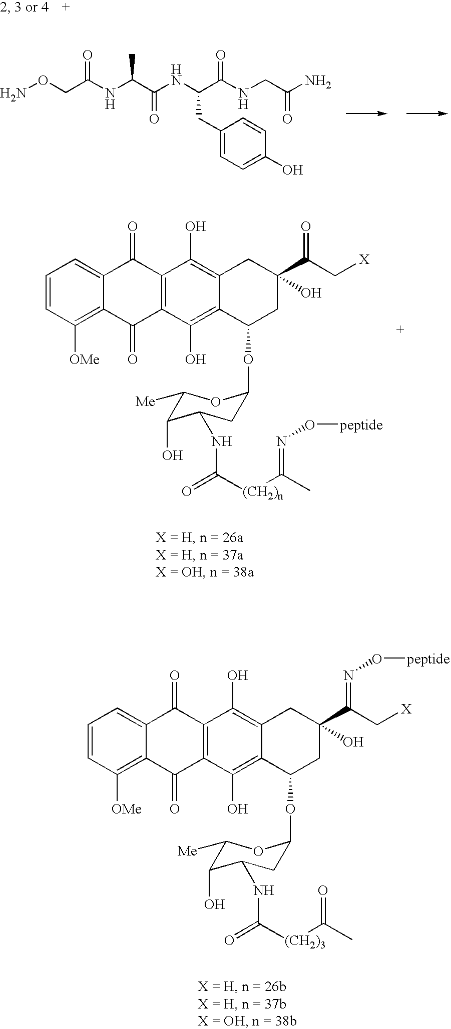

In a typical experiment 2.6 μmol of 5 and 3.9 μmol of keto derivative (2, 3, or 4) were dissolved in 1 ml citrate buffer (pH 2.6) or potassium acetate buffer (pH 4.0, 5.0, 6.0) and the reaction was monitored by HPLC (RP C-18 column, flow rate of I ml/min, linear gradient 5%-70% eluent B over 20 min, UV detection at 214 and 490 nm).

The pattern of MS fragmentation was studied through LC-MS (ES-MS) of the crude mixtures using the above gradient. Each isomer (obviously with the same molecular ion) showed a characteristic fragmentation pattern which allowed attribution of the structure; this was then confirmed through ¹H-NMR analysis of the isolated isomers.

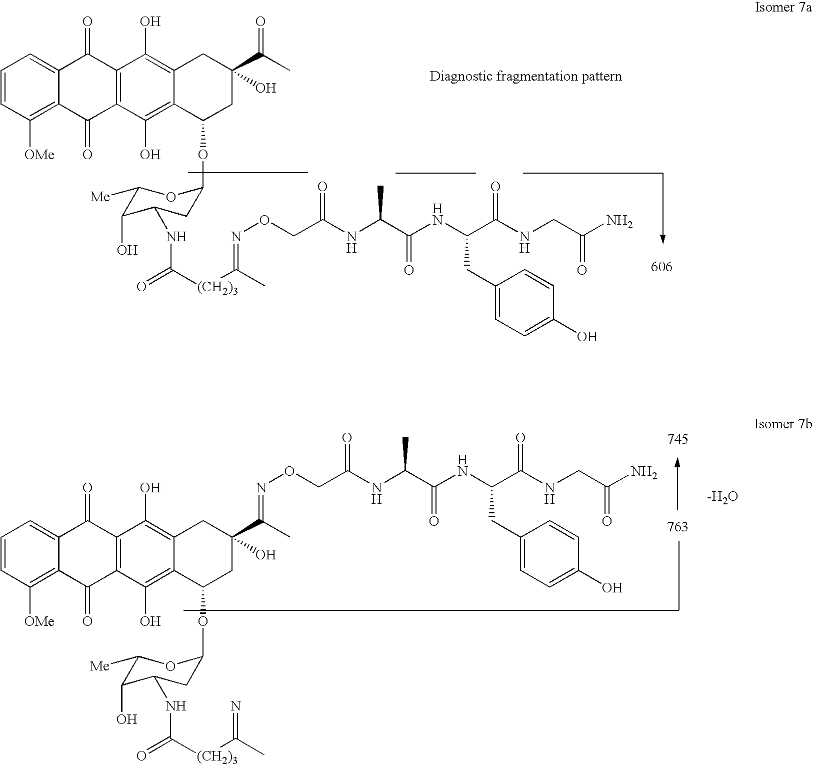

### Synthesis and Separation of the two Regioisomers 7a and 7b

A solution of peptide 5 (10 mg, 26 μmol) and daunorubicin derivative 2 (22 mg, 1.3 eq) in 5 ml potassium acetate buffer pH 4 was stirred at room temperature for 12 h. The solvents were distilled off in vacuo and the red residue submitted to preparative HPLC (Nucleosyl C18 column; 250×100 mm, 15 μm) using isocratic elution (5% eluent B, 5 min) followed by a linear gradient 5%-60% eluent B over 25 min. The fractions corresponding to the pure isomers were pooled; after lyophilization these yielded 3.5 mg of 7a and 3.5 mg of 7b (total yield 27%).

Isomer 7a: ¹H-NMR (DMSO-⁶d): 14.50 (s, 1H), 13.30 (s, 1H), 8,25 (t, 1H), 8.00 (d, 1H), 7.90 (m, 2H), 7.65 (m, 1H), 7.45 (m, 2H), 7.00 (d, 2H), 6.60 (d, 2H), 5.55 (s, 1H), 5.25 (s_{br}, 1H), 4.95 (dd, 1H), 4.75 (s_{br}, 1H), 4.40 (m, 1), 4.30 (m, 4), 4.20 (m, 1H), 4.00 (s, 3H), 3.75 (m, 2H), 3.40 (m, 1H), 3.00 (m, 3H), 2.70 (m, 1H), 2.25 (s, 3H), 2.15-2.05 (m, 4H), 1.80 and 1.75 (s, 3H), 1.75-1.50 (m, 5H), 1.45 (m, 1H), 1.15 (m, 6H). ES-MS analysis: [M+H⁺] m/z=1004, calculated for C₄₉H₅₈N₆O₁₂ 1003.

Isomer 7b: ¹H-NMR (DMSO-⁶d): 14.50 (s, 1H), 13.30 (s, 1H), 8.20 (m, 1H), 8.00 (m, 1H), 7.95 (m, 2H), 7.65 (m, 1H), 7.50 (m, 2H), 7.00 (d, 2H), 6.60 (d, 2H), 5.25 (m, 2H), 4.95 (m, 1H), 4.20 (s_{br}, 1H), 4.50-4.20 (m, 5H), 4.15 (m, 1H), 4.00 (s, 3H), 3.75 (s_{br}, 2H), 3.15-2.80 (m, 4H), 2.65 (m, 1H), 2.35 (m, 2H), 2.05 (m, 2H), 2.05 (s, 3H), 1.95 (s, 3H), 1.80 (m, 1H), 1.60 (m, 2H), 1.45 (m, 1H), 1.15 (m, 6H). ES-MS analysis: [M+H⁺] m/z=1004, calculated for C₄₉H₅₈N₆O₁₂ 1003.

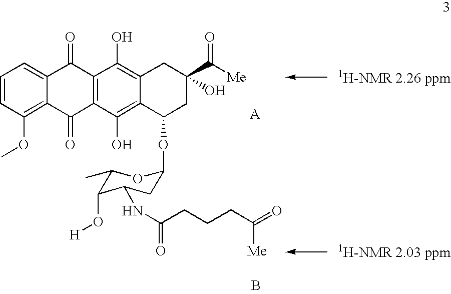

Formation of an oxime from a methyl ketone causes the methyl to shift toward higher fields in the corresponding ¹H-NMR spectrum. Comparison of the shifts of methyls A and B in 3 upon oxime formation confirms the structure assignment previously clone by LC-MS:

Isomer 7a: Me(A) 2.25 ppm, Me(B) 1.75 ppm.

Isomer 7b: Me(A) 1.95 ppm, Me(B) 2.05 ppm.

### pH Dependence of Regioselectivity in Oxime Formation

Regioselectivity for oxime formation was studied as a function of pH. Isomer ratios were evaluated by integration of peak area in the HPLC chromatogram of the crude mixtures obtained in the indicated experimental conditions.

As is apparent from FIG. 1, the desired regioisomer is favoured at higher pH. 5-Oxopentanoic acid gives better results than levulinic acid.

For the doxorubicin derivative 4 regioselectivity was maximal at pH 6, where the undesired regioisomer could not be detected (FIG. 2).

### Synthesis of Complex Peptide-Conjugates

As examples of the applicability of the method to larger peptides, including all the variety of side chains, were prepared: a) the conjugates of 2 and 3 with the 33-mer peptide 9, derivatized with aminooxyacetic acid on the ε-amino group of the C-terminal lysine; and b) a conjugate of 3 with a peptide containing a disulfide bridge (12).

### Synthesis of AEGEFALSETAKRWRLLFLRAGVGNAEDPAKGGK(COCH₂ONH₂)-CONH₂ (9)

The peptide was synthesized by Fmoc-t-Bu chemistry on a Millipore 9050 Plus synthesizer on 0.5 g of Fmoc-PAL-PEG-PS resin 0.19 meq/g (PE PerSeptive). The following side-chain protected amino acid derivatives were used: Fmoc-Tyr(t-Bu)-OH, Fmoc-Glu(Ot-Bu)-OH, Fmoc-Asp(Ot-Bu)-OH, Fmoc-Ser(t-Bu)-OH, Fmoc-Arg(Pmc)-OH, Fmoc-Lys(Boc)-OH, Fmoc-Lys(Alloc)-OH (for C-terminal Lys), Fmoc-Thr(t-Bu)-OH, Fmoc-Trp(Boc)-OH, and Fmoc-Asn(Trt)-OH. The N-terminal alanine was incorporated as the Boc derivative. The protected amino acids (1 eq) were preactivated with PyBOP (1 eq), HOBt (1 eq), and DIEA (2 eq) using a 5-fold excess of acylant over the resin amino groups. Coupling times were 60 min.

Cleavage of N^{ε}Allyloxycarbonyl Protecting Group of the C-Terminal Lys

The dried peptide resin was treated overnight with 10 ml of a solution of tetrakis(triphenylphosphine)palladium(0), 0.07M in CHCl₃ containing 5% acetic acid and 2.5% N-methylmorpholine. The resin was then drained and washed with DMF and repetitively with a solution 0.5% diethyldithiocarbamate and 0.5% DIEA in DMF.

Coupling of Boc-Aminooxyacetic Acid

The N^{ε}amino group of the C-terminal Lys was reacted with Boc-aminooxyacetic acid (1 eq), DIPC (1 eq) and HOBt (1 eq) for 2 h (5-fold excess of acylant). The resin was then washed with DMF, MeOH, diethyl ether and dried in vacuo.

Cleavage of the Peptide from the Resin

The peptide resin was treated with 20 ml of TFA 88%, phenol 5%, triisopropylsilane,2%, water 5% (Reagent B) for 2 h. The resin was filtered and rinsed with TFA. The TFA solution was added dropwise to screw cap centrifuge tubes containing cold MTBE with a TFA/MTBE ratio of 1/10; after centrifugation at 3200×g (30 min), the ether solution was removed and the peptide precipitate resuspended in 50 ml of MTBE: the process was repeated twice. The dried precipitate was dissolved in MeCN/water and lyophilized. The crude peptide was purified by preparative HPLC on a Waters Delta-Pak C-4 column (25×200 mm). In a typical run, the peptide (10 mg) was dissolved in water/0.1% TFA, loaded onto the preparative column and eluted with a linear gradient 20%-35% eluent B over 20 min at a flow rate of 30 ml/min. Fractions containing the desired peptide (98% pure) were pooled and lyophilized, yield 3 mg (30%). ES-MS analysis: calculated (average isotopic composition) 3768.2 Da, found 3768.4 Da.

### Synthesis of AEGEFALSETAKRWRLLFYRAGVGNAEDPAKGGK(COCH₂ON═Q) CONH₂ (10, Q=Daunorubicinone) and (11, Q=Doxorubicinone)

Both reactions were in aqueous buffer at pH 6, using a five-fold excess of 3 or 4. Target conjugates were smoothly produced in 24 h and isolated by preparative HPLC (RP C4, Phenomenex, 10×250 mm, linear gradient 15%-50% eluent B over 30 min). The yields were 43% and 34% respectively for 10 and 11.

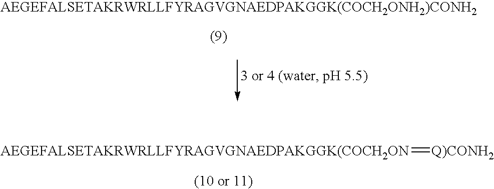

10: ES-MS analysis: [M+H⁺] m/z=4389, calculated for C₂₀₀H₂₉₅N₅₁O₆₁ 4389

11: ES-MS analysis: [M+H⁺] m/z=4406, calculated for C₂₀₀H₂₉₅N₅₁O₁₀ 4405

### Synthesis of

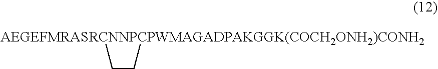

The peptide was synthesized by Fmoc-t-Bu chemistry as detailed in the previous Example. The following side-chain protected amino acid derivatives were used: Fmoc-Glu(Ot-Bu)-OH, Fmoc-Asp(Ot-Bu)-OH, Fmoc-Ser(t-Bu)-OH, Fmoc-Arg(Pmc)-OH, Fmoc-Lys(Boc)-OH, Fmoc-Lys(Alloc)-OH (C-terminal Lys), Fmoc-Trp(Boc)-OH, Fmoc-Cys(Trt)-OH and Fmoc-Asn(Trt)-OH. The N-terminal Ala was incorporated as the Boc derivative. The protected amino acids (1 eq) were preactivated with PyBOP (1 eq), HOBt (1 eq), and DIEA (2 eq) using a 5-fold excess of acylant over the resin amino groups. Coupling times were 60-90 min.

Cleavage of N^{ε}allyloxycarbonyl protecting group of the C-terminal. Lys and coupling of Boc-aminooxyacetic acid were performed as described in the previous Example.

Cleavage of the Peptide from the Resin and Purification

At the end of the assembly the resin was washed with DMF, MeOH, diethyl ether and dried in vacuo. The peptide resin was treated with TFA 88%, phenol 5%, triisopropylsilane 2%, water 5% (Reagent B) for 2 h, followed by work-up as previously described. The disulfide bridge was formed as described in Tam, J. P., Wu, C. -R., Liu, W. and Zhang, J. -W., J. Am. Chem. Soc., 1991, 113, 6657-6662: the crude peptide was stirred overnight in an aqueous solution of DMSO (15%, pH 7.2) at a concentration of 0.10-0.15 mg/ml; after completion of the reaction, the oxidized peptide was isolated by preparative HPLC.

The crude peptide was purified by preparative HPLC on a Waters Delta-Pak C-4 column (25×200 mm). In a typical run 130 ml of the oxidized peptide solution was acidified with TFA (0.1%), loaded onto the preparative column and eluted isocratically at 15% eluent B, followed by a linear gradient 15%-22% eluent B over 20 min at a flow rate of 30 ml/min. Fractions containing the desired peptide (98% pure) were pooled and lyophilized, yield 3 mg (20%). ES-MS analysis: calculated (average isotopic composition) 3022.4 Da, found 3022.3 Da.

### Synthesis of

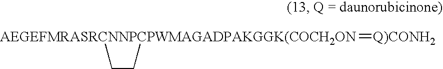

The reaction was run in aqueous buffer at pH 6.0, using a six-fold excess of 3. The target conjugate was produced in 5 days (regioisomer ratio 4:1) and isolated by HPLC on a semi-preparative Phenomenex C₄ (JUPITER) column (250×10 mm) by using a linear gradient 20%-45% of eluent B over 20 min at 5 ml/min (yield 23%). A second reaction, performed at pH 3.7, was completed in 24 h, but showed a regioisomer ratio of 1:1.

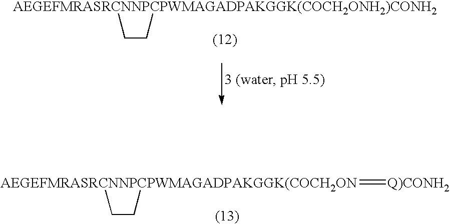

ES-MS analysis: calculated (average isotopic composition) 3643.5 Da, found 3643.2 Da.

1. A method of coupling an aminodrug and a peptide to form an aminodrug-peptide conjugate, the method comprising attaching a linker to an amino group of the drug, the linker including an aldehyde or ketone carbonyl group, and forming an oxime by reaction of the carbonyl group with an O-alkylhydroxylamine derivative of the peptide.

2. An aminodrug-peptide conjugate obtainable from the method as claimed in claim 1.

3. A conjugate as claimed in claim 2 wherein the aminodrug is a cytotoxic drug.

4. A conjugate as claimed in claim 2 or claim 3 wherein the aminodrug is a compound of formula (II) set out below: 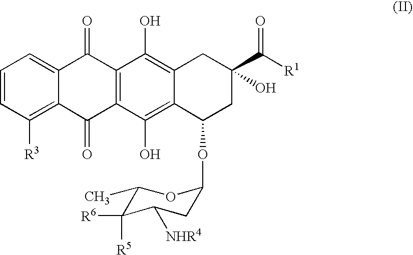
wherein: R¹ is —CH₃, —CH₂OH, —CH₂OCO(CH₂)₃CH₃ or —CH₂OCOCH(OC₂H₅)₂;
R³ is —OCH₃, —OH or —H;
R⁴ is —H, benzyl, cyanomethyl or —CH(CN)CH₂(OMe);
R⁵ is —OH, —OTHP or —H; and
R⁶ is —OH or —H; provided that R⁶ is not —OH when R⁵ is —OH or —OTHP;
and wherein the linker is attached at the amino group of the sugar moiety.

5. A conjugate as claimed in any one of claims 2 to 4 wherein the aminodrug is daunorubicin or doxorubicin.

6. A conjugate as claimed in any one of claims 2 to 5 wherein the linker is a group of formula (III) below: 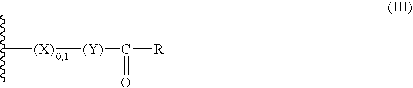
wherein X is selected from —CO—, —SO₂—, —SO₂NH—, —CO.O—, —CO.NH—, and —CR′R″— where each of R′ and R″ is independently selected from hydrogen and lower alkyl groups containing 1 to 10 carbon atoms;
Y is absent, or is an optionally substituted and/or interrupted alkylene group containing 1 to 6 carbon atoms, an optionally substituted and/or interrupted cycloalkylene group containing 3 to 7 carbon atoms, or an aromatic or heteroaromatic ring containing 2 to 10 carbon atoms; and
R is H or an optionally substituted and/or interrupted lower alkyl group containing 1 to 10 carbon atoms.

7. A conjugate as claimed in claim 6 wherein the linker is a group of formula (IVa) or (IVb):

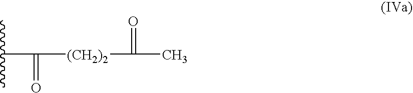

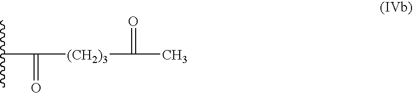

8. A conjugate as claimed in any one of claims 2 to 7 wherein the O-alkylhydroxylamine derivative of the peptide is a compound of formula (VI): 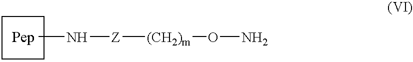
where 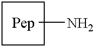
is an underivatised peptide with a free amino group; the group Z is selected from —CO— (forming an amide), —SO₂— (forming a sulfonamide), —CO.O— (forming a carbamate), —CO.NH— (forming a urea), and —SO₂.NH— (forming a sulfamide); and m is an integer from 1-6.

9. A conjugate as claimed in claim 8 wherein the compound of formula (VI) is selected from the following:

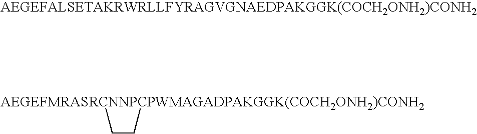

10. A pharmaceutical composition comprising a conjugate as claimed in any one of claims 2 to 9 in association with a pharmaceutically acceptable carrier.

11. The use of a conjugate as claimed in any one of claims 3 to 9 for the manufacture of a medicament for treating tumours.

12. A method for the treatment of tumours which comprises administering to a patient in need of such treatment an effective amount of a conjugate as claimed in any one of claims 3 to 9.
